(19) United States
(12) Patent Application Publication
(10) Pub. No.: US 2024/0060023 A1
(43) Pub. Date: Feb. 22, 2024

(54) CULTURE VESSEL, CULTURING SYSTEM, AND METHOD FOR PRODUCING CULTURE

(71) Applicants: Canon Medical Systems Corporation, Otawara-shi (JP); CANON KABUSHIKI KAISHA, Tokyo (JP)

(72) Inventors: Yosuke YAMAGUCHI, Kawasaki (JP); Ichiro HARADA, Kanagawa (JP); Yoji YAMAMOTO, Tokyo (JP); Yasuo SAKURAI, Nasushiobara (JP)

(73) Assignees: Canon Medical Systems Corporation, Otawara-shi (JP); CANON KABUSHIKI KAISHA, Tokyo (JP)

(21) Appl. No.: 18/448,278

(22) Filed: Aug. 11, 2023

(30) Foreign Application Priority Data
Aug. 22, 2022 (JP) 2022-131812

(51) Int. Cl. C12M 1/12; C12M 1/00; C12N 5/074; C12M 3/00

(52) U.S. Cl. CPC C12M 23/06; C12M 23/22; C12M 23/26; C12M 29/04; C12M 29/14; C12M 29/26; C12N 5/0696; C12M 23/44

(57) ABSTRACT

According to one embodiment, a culture vessel includes a rigid bottom part and a wall part. The wall part has a culture medium inlet and includes a deformable portion. The culture vessel allows the deformable portion to deform so as to change the capacity of the culture vessel when a pressure of the internal space of the culture vessel varies.

### CROSS-REFERENCE TO RELATED APPLICATION(S)

This application is based upon and claims the benefit of priority from prior Japanese Patent Application No. 2022-131812, filed Aug. 22, 2022, the entire contents of which are incorporated herein by reference.

### FIELD

Embodiments disclosed herein relate generally to a culture vessel, a culturing system, and a method for producing a culture.

### BACKGROUND

A low-cost establishment of induced pluripotent stem (iPS) cells requires a compact liquid feeding system that utilizes syringe pumps, pipe flow channels, etc., rather than manual procedures in a large-scale installation environment such as a clean room. For the sake of stable feeding of a cell suspension and a reagent to a culture vessel through a pipe flow channel, a pressure difference needs to be secured between the inlet and the outlet of the flow channel. In some instances, a culture vessel having a vent, for example, a vent filter or the like, as a means for suppressing pressure variations in its internal space is employed.

### BRIEF DESCRIPTION OF THE DRAWINGS

FIG. 1 is a diagram showing an exemplary culture vessel according to an embodiment.

FIG. 2 is a diagram showing an exemplary culturing system.

FIG. 3 is a diagram showing how the establishment and culturing of induced pluripotent stem cells proceed by use of the culturing system.

FIG. 4 is a diagram schematically showing a flow of blood in step S2.

FIG. 5 is a diagram schematically showing a flow of a wash liquid in step S4.

FIG. 6 is a diagram schematically showing a flow of an inducing-factor-containing culture medium in step S6.

FIG. 7 is a diagram schematically showing a flow of iPS cells in step S8.

FIG. 8 is a diagram showing a cross-section along the line VIII-VIII of the culture vessel shown in FIG. 1.

FIG. 9 is a diagram schematically showing a state where a culture medium has been supplied to the culture vessel shown in FIG. 8.

FIG. 10 is a diagram schematically showing a cross-section of a culture vessel according to a modification.

### DETAILED DESCRIPTION

In general, according to one embodiment, a culture vessel includes a rigid bottom part and a wall part. The wall part has a culture medium inlet and includes a deformable portion. The culture vessel allows the deformable portion to deform so as to change the capacity of the culture vessel when a pressure of the internal space of the culture vessel varies.

With reference to the drawings, embodiments of an apparatus and a method for the establishment of induced pluripotent stem cells will be described in detail.

### <Culture Vessel>

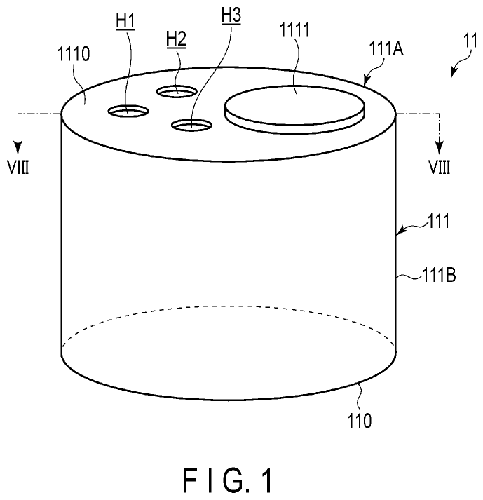

FIG. 1 is a diagram showing an exemplary culture vessel according to an embodiment. This culture vessel 11 changes its capacity according to a pressure variation in its internal space. As shown in FIG. 1, the culture vessel 11 includes a bottom part 110 and a wall part 111.

The bottom part 110 is rigid. Being rigid here means having an elastic modulus equal to or higher than 10 times the elastic modulus of a later-described deformable portion 1111, i.e., a soft portion.

The bottom part 110 preferably has a light transmitting property. For example, the bottom part 110 has visible-light transmitting properties. The bottom part 110 having light transmitting properties enables easy observation of an object being cultured. Preferably, the bottom part 110 is transparent.

In one example, the bottom part 110 is made of glass or resin. The resin is, for example, polycarbonate (PC) or polystyrene (PS). A preferred form of the bottom part 110 is made of polycarbonate (PC).

The wall part 111 is connected to the bottom part 110. The wall part 111 shown in FIG. 1 is constituted by a top part 111A facing the bottom part 110, and a side part 111B located between the bottom part 110 and the top part 111A.

The top part 111A includes a top main portion 1110 and the aforementioned deformable portion 1111.

The top main portion 1110 has a culture medium inlet H1, a gas inlet H2, a liquid outlet H3, and a hole H4. The deformable portion 1111 is placed at the hole H4.

In one example, the top main portion 1110 is rigid. For example, the same material as any of those discussed for the bottom part 110 may be used as the material of the top main portion 1110. The top main portion 1110 may or may not have a light transmitting property. Preferably, the top main portion 1110 has light transmitting properties. Such a top main portion 1110 enables easy observation of an object being cultured. More preferably, the top main portion 1110 has light transmitting properties for wavelengths in the visible-light spectrum.

The deformable portion 1111 is fixed at the position of the hole H4. The deformable portion 1111 thus closes the hole H4. The deformable portion 1111 is capable of deforming when the pressure of the internal space of the culture vessel 11 varies.

As one example, the deformable portion 1111 is an elastic member. Examples of the elastic member include an elastomer such as rubber.

The deformable portion 1111 preferably has an elastic modulus E1 in the range of 0.01 GPa to 0.1 GPa.

It is preferred that a ratio of the elastic modulus E1 of the deformable portion 1111 to an elastic modulus E2 of the bottom part 110, i.e., E1/E2, be 0.1 or lower.

Note that the “elastic modulus” here refers to a flexural modulus prescribed under JIS K7171:2022 (ISO 178:2019) or a tensile modulus prescribed under JIS K7161-1:2014 (ISO 527-1:2012). What has been noted for the elastic moduli E1 and E2 is preferably satisfied for at least one of the flexural modulus and the tensile modulus, and more preferably satisfied for both of the flexural modulus and the tensile modulus.

The side part 111b is rigid. For example, the same material as any of those discussed for the bottom part 110 may be used as the material of the side part 111B. The side part 111B may or may not have light transmitting properties. Preferably, the side part 111B has light transmitting properties. A more preferable form of the side part 111B is transparent.

The culture vessel 11 is intended for culturing an object which is, for example, cells such as animal cells and plant cells, tissue, or microorganisms. Examples of the animal cells include human cells. The animal cells may be, for example, stem cells. The stem cells may be, for example, induced pluripotent stem (iPS) cells, embryonic stem (ES) cells, or somatic stem cells. The object of culturing is preferably induced pluripotent stem cells. The object of culturing may take the form of a cell sheet. A resultant obtained by culturing such an object will be called a “culture”. The culture may be, for example, a cell population.

Preferably, the culture vessel 11 does not have an outlet for discharging a gas that fills the internal space of the culture vessel 11. A culture vessel 11 without such an outlet does not permit entry of microbes from outside, and therefore, it hardly causes contamination.

The culture vessel 11 has been described.

Note that while the top part 111A and the side part 111B shown in FIG. 1 are integrated, the top part 111A and the side part 111B may be detachable from each other.

Note also that while the top part 111A shown in FIG. 1 is constituted by the deformable portion 1111 and a rigid portion, i.e., the top main portion 1110, the top part 111A may be constituted solely by the deformable portion 1111.

Further, while the top part 111A shown in FIG. 1 has the deformable portion 1111, the deformable portion 1111 may be omitted from the top part 111A. The deformable portion 1111 may instead be provided at, for example, the side part 111B. That is, a structure where at least a portion of the wall part 111 has the deformable portion or portions 1111 serves the purpose.

Still further, while the wall part 111 shown in FIG. 1 is constituted by the top part 111A and the side part 111B, the wall part 111 may not be constituted by these parts. For example, the wall part 111 may have a dome shape. The gas inlet H2 and the liquid outlet H3 may be omitted as appropriate.

### <Culturing System>

The foregoing culture vessel 11 may be adopted in a culturing system. An exemplary culturing system including the foregoing culture vessel 11 will be described. Here, the description will assume the culturing system to be an arrangement for culturing induced pluripotent stem cells.

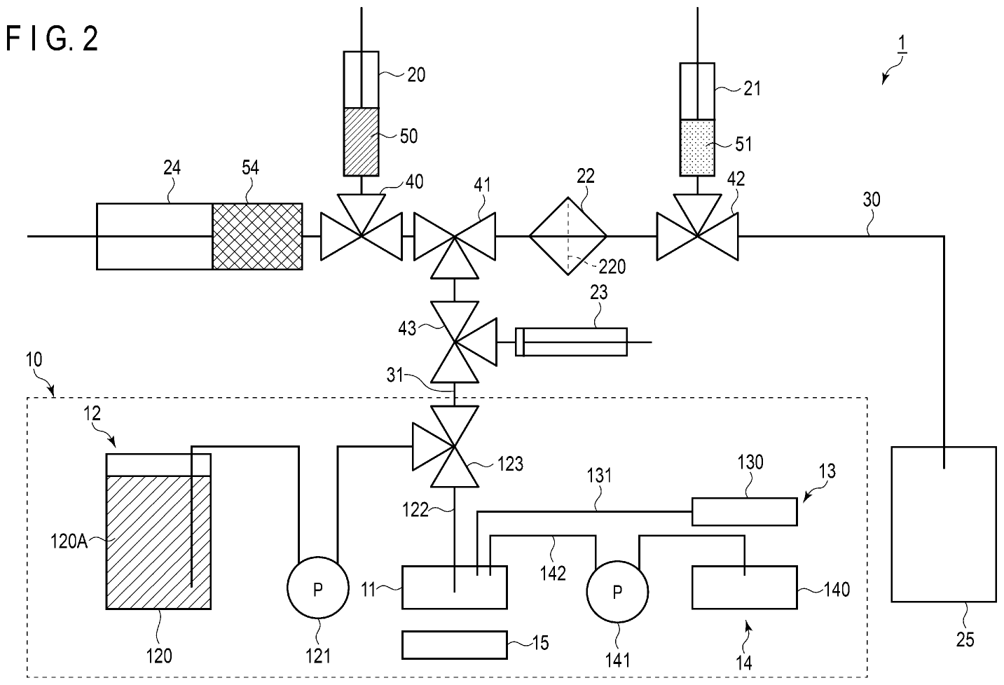

FIG. 2 is a diagram showing an exemplary configuration of the culturing system according to the embodiment. This culturing system 1 includes, as shown in FIG. 2, a culturing apparatus 10, a suspension discharger 20, an inducing factor supplier 21, a trap 22, an establisher 23, a wash liquid supplier 24, and a waste liquid receiver 25.

As shown in FIG. 2, the culturing apparatus 10, the suspension discharger 20, the inducing factor supplier 21, the trap 22, the establisher 23, the wash liquid supplier 24, and the waste liquid receiver 25 are each provided in association with a flow channel. In one example, the flow channel includes a first flow channel 30 and a second flow channel 31. As the components associated with the first flow channel 30, the wash liquid supplier 24, the suspension discharger 20, the trap 22, the inducing factor supplier 21, and the waste liquid receiver 25 are arranged in this order in the flow direction (from the upstream side to the downstream side). The first flow channel 30 receives an inflow suspension discharged from the suspension discharger 20 and an inflow inducing factor supplied from the inducing factor supplier 21. In the first flow channel 30, the suspension flows in a first direction, and the inducing factor flows in a second direction opposite the first direction. The second flow channel 31 is a path branching off from the first flow channel 30 at a position upstream of the trap 22 and has the establisher 23 and the culturing apparatus 10 downstream. Here, the position where the second flow channel 31 branches off from the first flow channel 30 is a position on the first flow channel 30 that is between the entrance for the suspension and the trap 22.

The suspension discharger 20 discharges a suspension 50 which contains target cells. The suspension 50 may be blood extracted from a human body and having target cells suspended therein, or a preservative solution in which the target cells are suspended. The target cells here refer to the cells to be reprogrammed by the inducing factors. The target cells may be cells obtained from blood or cells of another origin, as long as they are reprogrammable somatic cells. As the blood-derived target cells, mononuclear cells are used.

The suspension discharger 20 is constituted by, for example, an injector including a cylinder and a piston. The cylinder is adapted to contain the suspension 50 with an airtight configuration so that the suspension 50 is inhibited or prevented from being exposed to the ambient air. In this example, the tip of the cylinder is connected to the first flow channel 30 via a valve 40. The piston pushes out the suspension 50 contained in the cylinder.

The inducing factor supplier 21 supplies an inducing-factor-containing culture medium 51 to the trap 22. The inducing-factor-containing culture medium 51 includes inducing factors and a culture medium for establishing iPS cells. The inducing factors are employed for reprogramming the target cells and include gene(s) of Oct family, gene(s) of Klf family, and gene(s) of Myc family, or their respective gene products. In one example, Oct3/4 is used as the gene(s) of Oct family, Klf4 is used as the gene(s) of Klf family, and c-Myc or L-Myc is used as the gene(s) of Myc family. The inducing factors may also include gene(s) of Sox family and/or gene products thereof. As the gene(s) of Sox family, Sox2 may be used.

The inducing factor supplier 21 is constituted by, for example, an injector including a cylinder and a piston. The cylinder is adapted to contain the inducing-factor-containing culture medium 51 with an airtight configuration so that the culture medium 51 is inhibited or prevented from being exposed to the ambient air. The tip of the cylinder is connected to the first flow channel 30 via a valve 42. The piston pushes out the culture medium 51 contained in the cylinder.

The trap 22 is arranged at a position on the first flow channel 30 that is between the suspension discharger 20 and the inducing factor supplier 21. In other words, the trap 22 is arranged between the entrance for the suspension and the entrance for the inducing factor in the middle of the first flow channel 30. The trap 22 receives the suspension 50 discharged from the suspension discharger 20 and traps the target cells contained in the suspension 50. In one example, the trap 22 is constituted by a container including a filter 220 with meshes that can catch the target cells while permitting other substances having a smaller particle size than the target cells to pass through. The trap 22 has an airtight configuration so that its internal space is inhibited or prevented from being exposed to the ambient air.

The establisher 23 is connected to the second flow channel 31 so that the target cells and the inducing factor are supplied to the establisher 23 through the second flow channel 31. The establisher 23 allows the inducing factors supplied to the trap 22 to be introduced into the target cells that have been trapped by the trap 22 so as to establish induced pluripotent stem cells (iPS cells). The establisher 23 sends the established induced pluripotent stem cells to the culturing apparatus 10.

The establisher 23 is constituted by, for example, an injector including a cylinder and a piston. The cylinder is adapted to contain the inducing-factor-containing culture medium 51 that has been supplied from the inducing factor supplier 21 and passed through the trap 22, as well as the target cells that have been trapped by the trap 22. The cylinder has an airtight configuration so that the inducing-factor-containing culture medium 51 and the target cells are inhibited or prevented from being exposed to the ambient air. Also, this cylinder is adapted to contain the iPS cells established by the introduction of the inducing factor into the target cells. In this example, the tip of the cylinder is connected to the second flow channel 31 via a valve 43. The piston pushes out the iPS cells contained in the cylinder.

The wash liquid supplier 24 supplies a wash liquid 54 to the first flow channel 30 so that the trap 22 is washed by the wash liquid 54. The wash liquid 54 is, for example, a saline solution or the like, and a liquid that does very little damage to the target cells may be adopted as the wash liquid 54.

The wash liquid supplier 24 is constituted by, for example, an injector including a cylinder and a piston. The cylinder is adapted to contain the wash liquid 54 with an airtight configuration so that the wash liquid 54 is inhibited or prevented from being exposed to the ambient air. In this example, the tip of the cylinder is connected to the first flow channel 30 via the valve 40. The piston pushes out the wash liquid 54 contained in the cylinder.

The waste liquid receiver 25 is a container to receive waste liquid. The waste liquid receiver 25 receives, as the waste liquid, the suspension 50 and the wash liquid 54 that have passed through the trap 22. The waste liquid receiver 25 may be a flask, a bag, or any container that can receive and hold the waste liquid. The waste liquid receiver 25 has an airtight configuration so that its internal space is inhibited or prevented from being exposed to the ambient air.

The culturing apparatus 10 cultures the iPS cells established by the establisher 23. The culturing apparatus 10 includes the above-described culture vessel 11 and also a culture medium supplier 12, a gas supplier 13, a drainer 14, and an observation instrument 15. The culturing apparatus 10 has an airtight configuration so that the internal space of the culture vessel 11 is inhibited or prevented from being exposed to the ambient air. That is, in the culturing apparatus 10, the internal space of the culture vessel 11 is isolated from the atmosphere.

The culture medium supplier 12 supplies a culture medium for culturing a culturing object, which is iPS cells in this example, to the culture vessel 11. The culture medium supplier 12 includes a culture medium reservoir 120, a pump 121, a third flow channel 122, and a valve 123. The culture medium reservoir 120 is a container to reserve a culture medium 120A. One end of the third flow channel 122 is connected to the culture medium reservoir 120, and the other end of the third flow channel 122 is connected to the culture medium inlet H1 of the culture vessel 11. The third flow channel 122 is inserted into the culture medium inlet H1 so that no gap is formed between the third flow channel 122 and the edge of the culture medium inlet H1. As such, microbes do not enter through a gap which is otherwise formed between the third flow channel 122 and the edge of the culture medium inlet H1. The pump 121 is arranged at a position on the third flow channel 122 that is between the culture vessel 11 and the culture medium reservoir 120. The pump 121 suctions the culture medium 120A from the culture medium reservoir 120 and supplies the suctioned culture medium 120A to the culture vessel 11. The valve 123 is arranged on the third flow channel 122. To the valve 123, the second flow channel 31 is connected. The valve 123 is adapted to shift between permitting and stopping of the supply of the induced pluripotent stem cells established by the establisher 23 to the third flow channel 122. The valve 123 is also adapted to shift between permitting and stopping of the supply of the culture medium from the culture medium reservoir 120 to the culture vessel 11.

The gas supplier 13 supplies a gas for culturing the culturing object to the culture vessel 11. This gas is, for example, a carbon-dioxide-containing gas. The gas supplier 13 includes a gas container 130 and a fourth flow channel 131. The gas container 130 is a container to reserve the gas. One end of the fourth flow channel 131 is connected to the gas container 130, and the other end of the fourth flow channel 131 is connected to the gas inlet H2 of the culture vessel 11. The fourth flow channel 131 is inserted into the gas inlet H2 so that no gap is formed between the fourth flow channel 131 and the edge of the gas inlet H2. As such, microbes do not enter through a gap which is otherwise formed between the fourth flow channel 131 and the edge of the gas inlet H2.

Note that the other end of the fourth flow channel 131 may instead be connected to a portion of the third flow channel 122 that is between the valve 123 and the culture vessel 11. Such an arrangement can omit the gas inlet H2.

The drainer 14 drains the culture medium from the culture vessel 11. The drainer 14 includes a drainage receiver 140, a pump 141, and a fifth flow channel 142. The drainage receiver 140 is a container to receive the culture medium discharged from the culture vessel 11. One end of the fifth flow channel 142 is connected to the drainage receiver 140, and the other end of the fifth flow channel 142 is connected to the liquid outlet H3 of the culture vessel 11. The fifth flow channel 142 is inserted into the liquid outlet H3 so that no gap is formed between the fifth flow channel 142 and the edge of the liquid outlet H3. As such, microbes do not enter through a gap which is otherwise formed between the fifth flow channel 142 and the edge of the liquid outlet H3. The pump 141 is arranged at a position on the fifth flow channel 142 that is between the drainage receiver 140 and the culture vessel 11. The pump 141 suctions the culture medium from the culture vessel 11 and sends the suctioned culture medium to the drainage receiver 140.

The observation instrument 15 is a device for observing the culturing object in the culture vessel 11. Examples of the observation instrument 15 include a microscope such as an optical microscope, and an imaging device. In one example, the observation instrument 15 enables phase-contrast observation of the culturing object. The observation instrument 15 may or may not include an observation light source.

The culturing apparatus 10 has been described.

The first flow channel 30 has multiple valves including the aforementioned valves 40 and 42 and also a valve 41. The valve 40 is arranged at or near one end (an upstream side) of the first flow channel 30, and the suspension discharger 20 and the wash liquid supplier 24 are connected to the valve 40. The valve 40 is adapted to shift between permitting and stopping of the supply of the suspension from the suspension discharger 20 to the first flow channel 30, and also to shift between permitting and stopping of the supply of the wash liquid from the wash liquid supplier 24 to the first flow channel 30. The valve 41 is arranged at the position where the second flow channel 31 branches off from the first flow channel 30. The valve 42 is arranged at or near the other end (a downstream side) of the first flow channel 30, and the inducing factor supplier 21 is connected to the valve 42. The valve 42 is adapted to shift between permitting and stopping of the supply of the inducing-factor-containing culture medium 51 to the first flow channel 30. The second flow channel 31 has the aforementioned valve 43. To the valve 43, the establisher 23 is connected. The valve 43 is adapted to shift between permitting and stopping of the supply of the inducing-factor-containing culture medium 51 that has passed through the trap 22 and the target cells that have been trapped by the trap 22, to the establisher 23.

The valves 40 to 43, as well as the valve 123, may each be a two-way valve, a three-way valve, or any type of valve that can open and close the associated channels. As one example, the description will assume each of the valves 40 to 43 and 123 to be a three-way valve adapted as a channel switching valve. A three-way valve is a mechanical component constituted by a body element having three openings and a valving element adapted to open two of these openings while closing the remaining one opening. The valving elements in the valves 40 to 43 and 123 may each perform channel switching actions under a manual and/or an electromagnetic control.

The first flow channel 30, the second flow channel 31, the third flow channel 122, the fourth flow channel 131, and the fifth flow channel 142 are each constituted by, for example, an airtight flexible tubular member such as a vinyl or plastic tube. Each liquid flowing through the first flow channel 30, the second flow channel 31, the third flow channel 122, the fourth flow channel 131, and the fifth flow channel 142 is thus inhibited or prevented from being exposed to the ambient air.

### <Operations of Culturing System>

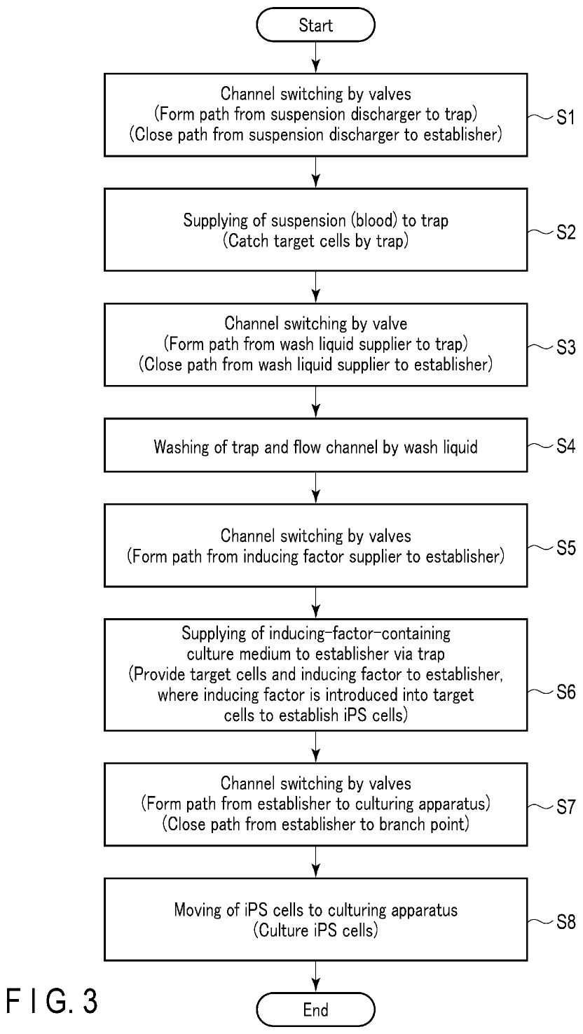

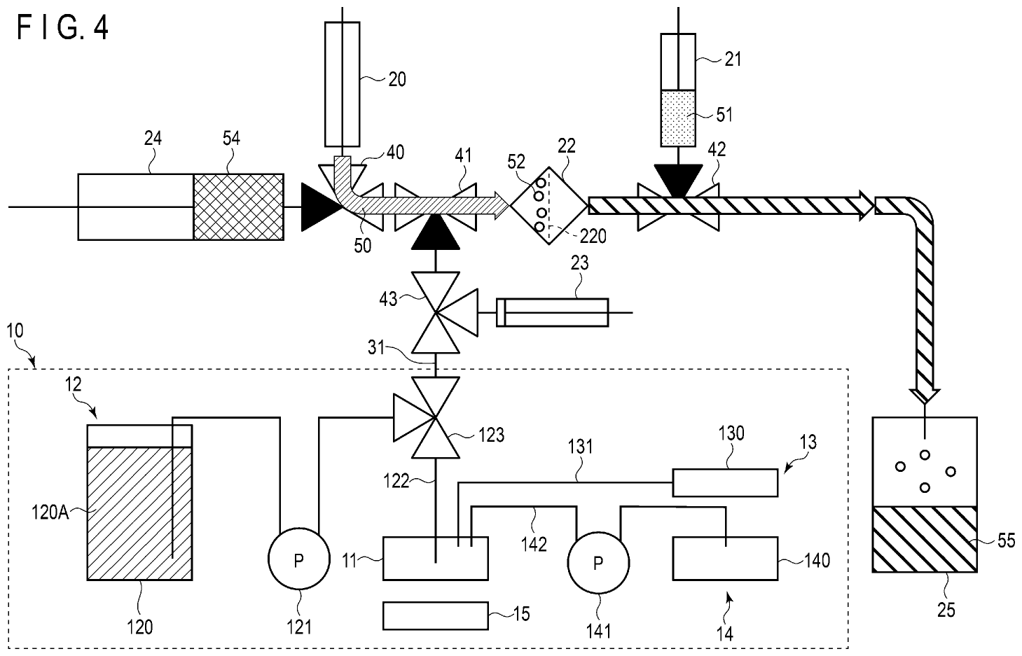

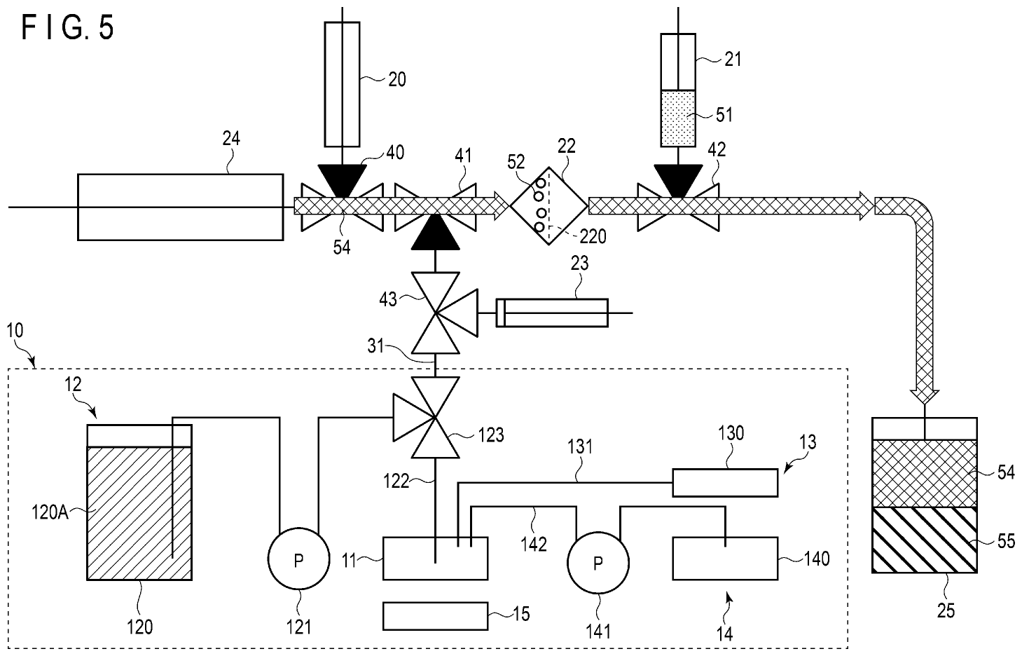

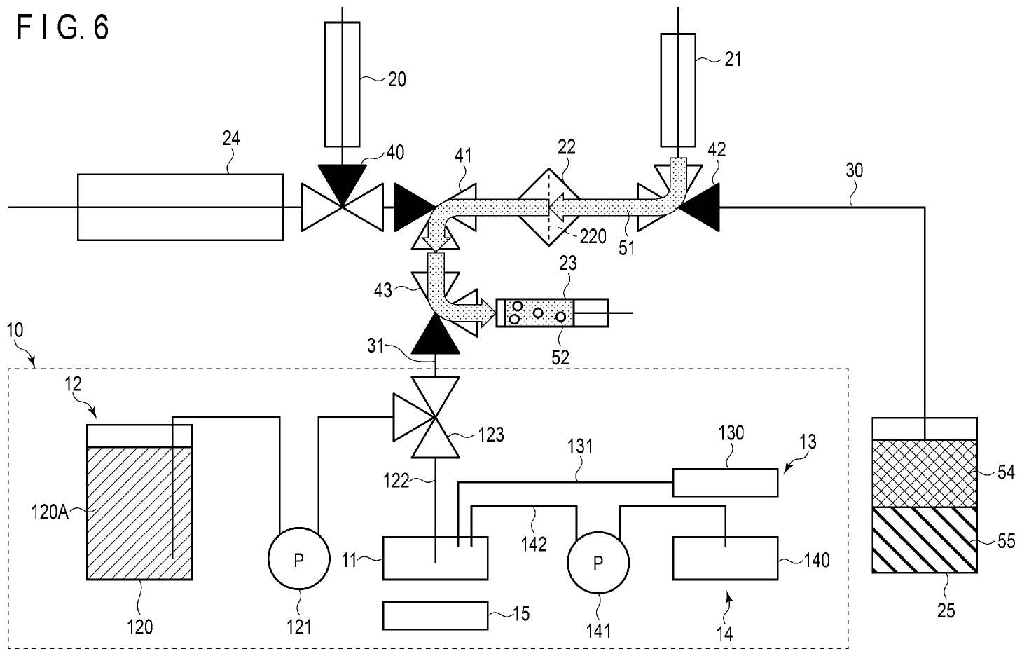

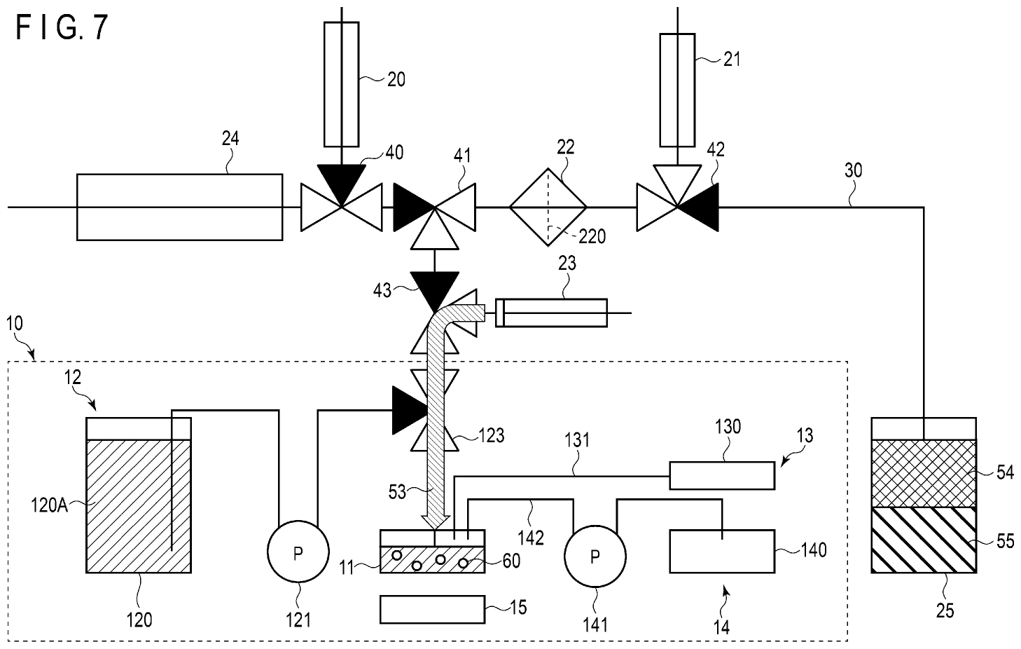

Exemplary operations of the culturing system 1 configured as above will be described. FIG. 3 is a diagram showing how the establishment and culturing of the iPS cells proceed by use of the culturing system 1. FIG. 4 is a diagram schematically showing a flow of blood in step S2. FIG. 5 is a diagram schematically showing a flow of the wash liquid in step S4. FIG. 6 is a diagram schematically showing a flow of the inducing-factor-containing culture medium in step S6. FIG. 7 is a diagram schematically showing a flow of the iPS cells in step S8. Note that the example shown in FIG. 3 assumes that the suspension is blood collected from a donor. It will also be assumed that, at the start of the operation shown in FIG. 3, the suspension discharger 20 retains the blood, the inducing factor supplier 21 retains the inducing-factor-containing culture medium, and the wash liquid supplier 24 retains the wash liquid, each in an amount corresponding to one operation.

As shown in FIG. 3, the channel switching by the valves 40, 41, and 42 is performed first (step 31). More specifically, a path from the suspension discharger 20 to the trap 22 is formed while a path from the suspension discharger 20 to the establisher 23 is closed. For this, an operator operates the valve 40 so that its valving element opens the opening to the suspension discharger 20 (which may be called a “blood supplying opening”) and the opening to the first flow channel 30 (which may be called a “discharging opening”) and closes the opening to the wash liquid supplier 24 (which may be called a “wash liquid supplying opening”). Also, the operator operates the valve 41 so that its valving element opens the opening to the suspension discharger 20 (which may be called a “blood and wash liquid supplying opening”) and the opening to the trap 22 (which may be called a “trap side opening”) and closes the opening to the establisher 23 (which may be called an “establisher side opening”). The operator further operates the valve 42 so that its valving element opens the opening to the trap 22 (which may be called a “trap side opening”) and the opening to the waste liquid receiver 25 (which may be called a “waste liquid supplying opening”) and closes the opening to the inducing factor supplier 21 (which may be called an “inducing factor supplying opening”). Note that if the initial states of the valves have already ensured that the path from the suspension discharger 20 to the trap 22 is secured and that the path from the suspension discharger 20 to the establisher 23 is closed, step S1 may be omitted. Note also that the drawings indicate each of the valves 40, 41, 42, 43, and 123 using three triangle symbols, and these triangle symbols represent three respective openings of the three-way valve. Here, a white triangle indicates an opening opened by the corresponding valving element, and a black triangle indicates an opening closed by the corresponding valving element.

After step S1, the suspension 50 (blood) containing target cells is supplied to the trap 22 (step S2). In step S2, the suspension discharger 20 discharges the suspension 50 in a forward direction through the first flow channel 30 to the trap 22. The forward direction is defined as a direction in which the suspension 50 flows toward the waste liquid receiver 25. More specifically, the operator operates the suspension discharger 20 so that the suspension 50 retained in the suspension discharger 20 is discharged into the first flow channel 30. The suspension 50 contains the target cells required for preparing iPS cells, and in this example, the suspension 50 is retained in the suspension discharger 20 in an amount corresponding to one operation. Thus, all of the suspension 50 retained in the suspension discharger 20 may be discharged. The suspension 50 discharged into the first flow channel 30 flows and enters the trap 22. Among the components (substances) contained in the suspension 50 flowing into the trap 22, the components having a particle size larger than the mesh opening of the filter 220 are caught by the filter 220, while the components having a particle size smaller than the mesh opening of the filter 220 are permitted to pass through. The filter 220 here may be a filter with meshes that can catch, for example, components each having a size equivalent to a white blood cell. Each target cell 52, having a particle size larger than the opening of such meshes, is caught by the filter 220. Red blood cells, etc., not handled as target cells in this example, each have a particle size smaller than the mesh opening, and therefore pass through the filter 220. The blood components that have passed through the trap 22 are received and held by the waste liquid receiver 25 as a part of a waste liquid 55.

After step S2, the channel switching by the valve 40 is performed (step S3). More specifically, a path from the wash liquid supplier 24 to the trap 22 is formed while a path from the wash liquid supplier 24 to the establisher 23 is closed. For this, the operator operates the valve 40 so that its valving element opens the wash liquid supplying opening and the discharging opening and closes the blood supplying opening.

After step S3, the trap 22 is washed by the wash liquid 54 (step S4). In step S4, the wash liquid supplier 24 causes the wash liquid 54 to wash away the components remaining in the first flow channel 30 and the trap 22, except the target cells, into the waste liquid receiver 25. More specifically, the operator in step S4 operates the wash liquid supplier 24 so that the wash liquid 54 retained in the wash liquid supplier 24 is discharged into the first flow channel 30. Since the wash liquid 54 is retained in the wash liquid supplier 24 in an amount corresponding to one operation, all of the wash liquid 54 retained in the wash liquid supplier 24 may be discharged. The wash liquid 54 discharged into the first flow channel 30 causes the unwanted components remaining in the first flow channel 30 and the trap 22 other than the target cells to flow into the waste liquid receiver 25. In one example, components such as plasma, red blood cells, and platelets mainly constitute the unwanted blood components. Here, by adopting a configuration of advancing and retracting the piston of the wash liquid supplier 24 to repeat the discharge and suction of the wash liquid 54, it is possible to efficiently remove and sweep away the unwanted blood components sticking to the first flow channel 30 or the trap 22. Further, the culturing system 1 may be adapted to incline itself in conjunction with the discharge and suction of the wash liquid 54 so that the unwanted blood component can be efficiently washed away.

After step S4, the channel switching by the valves 41, 42, and 43 is performed (step S5). More specifically, a path from the inducing factor supplier 21 to the establisher 23 is formed. For this, the operator operates the valve 41 so that its valving element opens the opening to the trap 22 and the opening to the establisher 23 and closes the opening to the wash liquid supplier 24. Also, the operator operates the valve 42 so that its valving element opens the opening to the inducing factor supplier 21 and the opening to the trap 22 and closes the waste liquid supplying opening. Together, the operator operates the valve 43 so that its valving element opens the opening to the valve 41 (which may be called a “branch valve side opening”) and the opening to the establisher 23 (which may be called an “establisher side opening”) and closes the opening to the culturing apparatus 10 (which may be called a “culturing apparatus side opening”).

After step S5, the inducing-factor-containing culture medium 51 is supplied to the establisher 23 via the trap 22 (step S6). In step S6, the inducing factor supplier 21 supplies the inducing-factor-containing culture medium 51 to the trap 22 in the direction opposite the forward direction through the first flow channel 30, so that the inducing-factor-containing culture medium 51 together with the target cells 52 are supplied to the establisher 23 via the second flow channel 31. More specifically, the operator in step S6 operates the inducing factor supplier 21 to discharge the inducing-factor-containing culture medium 51 retained in the inducing factor supplier 21 into the first flow channel 30. The inducing-factor-containing culture medium 51 is retained in the inducing factor supplier 21 in an amount corresponding to one operation, and therefore, all of the inducing-factor-containing culture medium 51 retained in the inducing factor supplier 21 may be discharged. The discharged inducing-factor-containing culture medium 51 flows into the trap 22 from the direction opposite the flow direction of the suspension 50 in step S2. The inducing-factor-containing culture medium 51 that has entered the trap 22 passes through the filter 220 and flows, together with the target cells 52 caught by the filter 220, into the establisher 23 via the second flow channel 31. Note that the piston of the establisher 23 may be pulled to the limit beforehand in order to accommodate the inducing-factor-containing culture medium 51 and the target cells 52.

In the establisher 23, the inducing factor is introduced into the target cells 52 and iPS cells are established from the target cells. The inducing factor may be provided in a variety of forms. For example, the inducing factor may be integrated into vectors and provided. The vectors here are not limited, and viral vectors such as a Sendai virus vector and a retroviral vector, or non-viral vectors such as a plasmid may be suitably adopted. In an example where an inducing-factor-integrated Sendai virus vector is used, the Sendai virus vector and the target cell are brought into contact with each other so that the inducing factor is introduced into this target cell. A gene product of the inducing factor is then generated. The gene product induces the reprogramming of the target cell, thereby giving birth to an iPS cell which is an undifferentiated cell having a pluripotency and a proliferating ability. The thus-obtained iPS cells are left to grow under the presence of the culture medium for approximately an hour. In this manner, the iPS cells are established. During the establishing process, the establisher 23 may be suitably maintained at approximately 37° C. and 5% CO₂.

For the purpose of enhancing the accuracy of iPS cell establishment in the establisher 23, a container of a relatively small capacity may be suitably used as the cylinder of the establisher 23. This can increase the likelihood of the target cells and the inducing factor or the inducing-factor-integrated vector contacting each other, and consequently, increase the likelihood of the iPS cells being successfully established.

After step S6, the channel switching by the valves 43 and 123 is performed (step S7). More specifically, a path from the establisher 23 to the culturing apparatus 10 is formed. For this, the operator operates the valve 43 so that its valving element opens the establisher side opening and the culturing apparatus side opening and closes the branch valve side opening. Together, the operator operates the valve 123 so that its valving element opens the opening to the valve 43 (which may also be called an “establisher side opening”) and the opening to the culturing apparatus 10 (which may also be called a “culturing apparatus side opening”) and closes the opening to the culture medium reservoir 120 (which may be called a “culture medium supplying opening”).

After step S7, the iPS cells are moved to the culture vessel 11 (step S8). More specifically, the operator in step S7 operates the establisher 23 so that a suspension 53, i.e., the suspension in which the iPS cells are suspended, is discharged from the establisher 23 into the second flow channel 31. The iPS cell suspension 53 discharged into the second flow channel 31 moves to the culture vessel 11. Here, iPS cells 60, i.e., the iPS cells that have been moved to the culture vessel 11, undergo a culturing process there. During the culturing process, the culture vessel 11 may be suitably maintained at a room temperature of approximately 37° C. For culturing, the culture medium 120A may be provided from the culture medium supplier 12 to the culture vessel 11 suitably and as needed. For example, the operator may operate the valve 123 so that its valving element opens the culture medium supplying opening and the culturing apparatus side opening and closes the establisher side opening, and operate the pump 121 so that an appropriate amount of the culture medium 120A is supplied from the culture medium reservoir 120 to the culture vessel 11. The culture medium 120A may be constantly or intermittently supplied to the culture vessel 11.

For culturing, a prescribed gas may also be provided from the gas supplier 13 to the culture vessel 11 suitably and as needed.

For culturing, further, the culture medium 120A may be drained to the drainage receiver 140 from the culture vessel 11 suitably and as needed. For example, the operator may operate the valve 123 so that its valving element closes the culture medium supplying opening, and operate the pump 141 so that the culture medium 120A is drained to the drainage receiver 140 from the culture vessel 11.

In order to have the iPS cells 60 settle in the culture vessel 11, a coating agent may be applied to the culture vessel 11 prior to step S8. As a concrete example, a coating agent may be applied to the inside of the bottom part 110 of the culture vessel 11. The coating agent is preferably made of cell adhesion molecules such as laminin protein, vitronectin, etc. The culture vessel 11 can thus form a scaffold for the iPS cells 60 supplied from the establisher 23 and allow for the stable culturing of the iPS cells 60.

Note that the coating agent is not required to be applied to the culture vessel 11 in advance. For example, the operator may provide the coating agent to the culture vessel 11 using an injector at the time of supplying the iPS cells 60 from the establisher 23 to the culture vessel 11. The coating agent may be provided concurrently with, prior to, or after supplying the iPS cells 60 from the establisher 23 to the culture vessel 11.

Preferably, the culturing apparatus 10 is detachable from the culturing system 1. As one exemplary form, the culturing apparatus 10 can be separated from the culturing system 1 by pulling out the second flow channel 31 from the valve 123. Detachment of the culturing apparatus 10 from the culturing system 1 enables culturing of the iPS cells using a smaller site.

Note that the culturing apparatus 10 may itself be employed as a culturing system. In such instances, the valve 123 may be omitted.

It is preferable that at least one of the suspension discharger 20, the inducing factor supplier 21, and the establisher 23 be detachable from the culturing system 1. Also, the culture vessel 11 is preferably detachable from the third flow channel 122.

At least one of the gas supplier 13, the drainer 14, and the observation instrument 15 may be omitted.

By the process as described above, the establishment and culturing of iPS cells by use of the culturing system 1 are completed. According to such a process, the culture mentioned above can be produced.

Supposing that the culture medium supplier 12 supplies the culture medium to a culture vessel that is not furnished with the above-described deformable portion 1111, the pressure in the internal space of the culture vessel increases as the culture medium is supplied. That is, the difference between the pressure of the culture medium at the downstream of the pump 121 and the pressure in the internal space of the culture vessel, which is being increased, becomes small. This could result in an event where the culture medium is not properly supplied to the culture vessel, or an event where the culture medium once supplied to the culture vessel flows backward to the culture medium supplier 12 upon the pump 121 stopping its action.

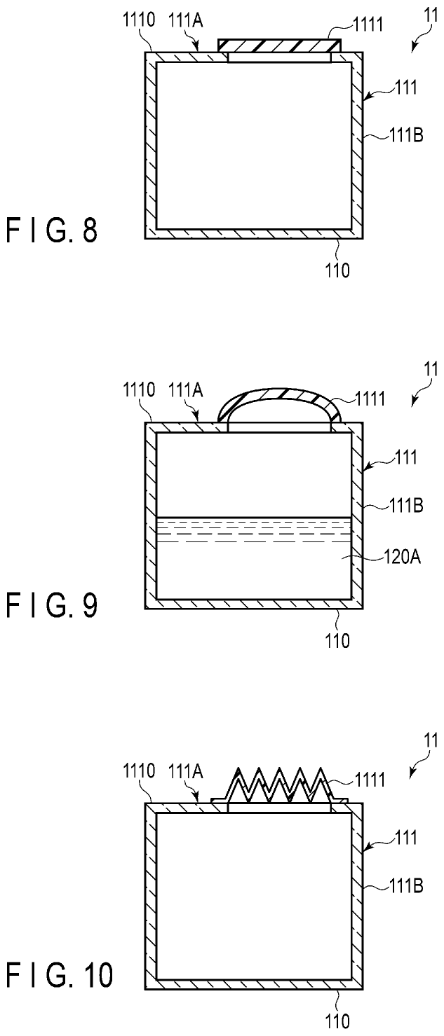

In contrast, with the culture vessel 11 described above, pressure variations in the internal space can be suppressed. A description will be given of the capability of suppressing the internal space pressure variations with reference to FIGS. 8 and 9.

FIG. 8 is a diagram showing a cross-section along the line VIII-VIII of the culture vessel 11 shown in FIG. 1. FIG. 9 is a diagram schematically showing a state where the culture medium has been supplied to the culture vessel 11 shown in FIG. 8.

For the culture vessel 11 in the state shown in FIG. 8, supplying of the culture medium 120A increases the pressure in the internal space of the culture vessel 11 and causes the deformable portion 1111 to deform as shown in FIG. 9. This deformation increases the capacity of the culture vessel 11. Accordingly, the pressure increase in the internal space of the culture vessel 11 is relieved and suppressed. As such, even if a further amount of the culture medium 120A is supplied to the culture vessel 11, a back-flow of the culture medium 120A, or the hindrance of the proper supplying of the culture medium 120A from the culture medium supplier 12 to the culture vessel 11, does not occur.

As one example, it will be assumed that the culture vessel 11 has a configuration in which: the bottom part 110 has a circular shape with a diameter of 35 mm; the side part 111B has a height of 30 mm; the deformable portion 1111 is made of rubber having an elastic modulus E1 of 0.1 GPa; and the bottom part 110, the side part 111B, and the top main portion 1110 are made of polycarbonate having an elastic modulus E2 of 2.3 GPa. In the state where this culture vessel 11 contains the culture medium in an amount up to the height of 10 mm, the internal space accommodates 19.2 cc of air. If 4.8 cc of the culture medium is further supplied to the culture vessel 11, the deformable portion 1111 deforms and the capacity of the culture vessel 11 increases by 4.2 cc. As a result, the volume of air in the internal space becomes 14.4 cc. On the assumption that the product of an internal space pressure and a capacity is constant before and after the supplying of the culture medium, the internal space pressure after the supplying of the culture medium is 1.03 times higher than the internal space pressure before the supplying of the culture medium.

However, in the case of using a culture vessel that is identical with this culture vessel 11 except that the deformable portion 1111 is not furnished, the internal space pressure after the supplying of the culture medium is 1.33 times higher than the internal space pressure before the supplying of the culture medium.

As understood from this, the culture vessel 11 described above can suppress the pressure increase occurring in its internal space due to the supplying of the culture medium. The culture vessel 11 described above can also equalize the pressure in its internal space and the pressure outside it.

The culture vessel 11 described above has the bottom part 110 which is rigid. The culture vessel 11 having such a structure allows for easy observation of a culturing object through an observation instrument such as a microscope.

In the culture vessel 11 shown in FIG. 1, the top part 111A includes the top main portion 1110 and the deformable portion 1111. With the top main portion 1110 having light transmitting properties, observation of a culturing object is easy as compared to the structure where the top part 111A is constituted only by the deformable portion 1111.

In some instances, a culture vessel is formed with a vent, for example, a vent filter or the like, in order to suppress the pressure increase as discussed above. Unfortunately, a vent filter requires, as its structure, holes each having a size of at least 0.2 μm for securing stable ventilation. As such, a vent filter cannot block out the entry of external microbes such as mycoplasma, and it cannot prevent the occurrence of contamination.

According to the culturing apparatus 10 and the culturing system 1 described above, the internal space of the culture vessel 11 is isolated from the atmosphere. Therefore, even the microbes that can pass through the holes having a size of 0.2 μm or smaller are barred from entry into the culture vessel 11. The culturing apparatus 10 and the culturing system 1 described above thus hardly create a contamination event.

Moreover, the culturing system 1 described above can realize the low-cost cell culture.

### <Modifications of Culture Vessel>

FIG. 10 is a diagram schematically showing a cross-section of the culture vessel according to a modification. The culture vessel 11 shown in FIG. 10 is identical with the culture vessel shown in FIG. 10 except that the deformable portion 1111 is recessed toward the internal space of the culture vessel 11. This culture vessel 11 shown in FIG. 10 has the deformable portion 1111 recessed toward the internal space in the state before use, for example, before receiving the supply of the culture medium or the culturing object. Also in the culture vessel 11 shown in FIG. 10, the deformable portion 1111 deforms to increase the capacity of the culture vessel 11 according to a pressure produced along with the supplying of the culture medium 120A to the culture vessel 11. Therefore, this culture vessel 11 can likewise suppress the pressure increase occurring in its internal space due to the supplying of the culture medium.

It is preferable for such modifications that the deformable portion 1111 include one surface having a surface area S1 and a projected area S2 where S1 is larger than S2. For example, in the case of the culture vessel 11 shown in FIG. 10, the projected area S2 represents an area of the subject surface orthogonally projected onto a surface of the bottom part 110.

Preferably, the ratio of the surface area S1 to the projected area S2, i.e., S1/S2, is 2 or greater.

Note that in this culture vessel 11, the deformable portion 1111 may or may not be an elastic member.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. In relation to the foregoing embodiments, etc., the following disclosures are additionally given, which set forth some of the various aspects of the inventions and alternative features thereof.

### (Supplementary Note 1)

A culture vessel comprising:
a rigid bottom part; and
a wall part having a culture medium inlet and comprising a deformable portion,
wherein the culture vessel is configured to allow the deformable portion to deform so as to change a capacity of the culture vessel when a pressure of an internal space of the culture vessel varies.

### (Supplementary Note 2)

The bottom part may have light transmitting properties. The bottom part may be transparent.

### (Supplementary Note 3)

The bottom part may be made of glass or resin. The resin may be polycarbonate.

### (Supplementary Note 4)

The deformable portion may be an elastic member. The elastic member may be an elastomer such as rubber.

### (Supplementary Note 5)

The deformable portion may have an elastic modulus E1 in the range of 0.01 GPa to 0.1 GPa.

### (Supplementary Note 6)

A ratio E1/E2 of the elastic modulus E1 of the deformable portion to an elastic modulus E2 of the bottom part may be 0.1 or lower.

### (Supplementary Note 7)

The deformable portion may be recessed toward the internal space.

### (Supplementary Note 8)

The deformable portion may have one surface whose surface area S1 and projected area S2 satisfy a relationship wherein S1 is larger than S2. A ratio S1/S2 of the surface area S1 to the projected area S2 may be 2 or greater.

### (Supplementary Note 9)

The wall part may comprise a top part facing the bottom part, and a side part between the bottom part and the top part.

The top part may comprise the deformable portion.

### (Supplementary Note 10)

The top part may comprise a top main portion and the deformable portion. The top main portion may be rigid. The top main portion may comprise a culture medium inlet, a gas inlet, a liquid outlet, and a hole. The deformable portion may be fixed at the position of the hole.

### (Supplementary Note 11)

The top main portion may have light transmitting properties. The top main portion may be transparent.

### (Supplementary Note 12)

The side part may have light transmitting properties. The side part may be transparent.

### (Supplementary Note 13)

An object of culturing by the culture vessel may be a cell such as an animal cell or a plant cell, tissue, or a microorganism. The animal cell may be a human cell. The animal cell may be a stem cell. The stem cell may be an induced pluripotent stem (iPS) cell, an embryonic stem (ES) cell, or a somatic stem cell. The object of culturing may be an iPS cell.

### (Supplementary Note 14)

The culture vessel may be without an outlet for discharging a gas that fills the internal space.

### (Supplementary Note 15)

A culturing system comprising:
a culture vessel comprising a rigid bottom part and a wall part, the wall part having a culture medium inlet and comprising a deformable portion, the culture vessel being configured to allow the deformable portion to deform so as to change a capacity of the culture vessel when a pressure of an internal space of the culture vessel varies; and
a culture medium supplier connected to the culture medium inlet and configured to supply a culture medium into the culture vessel via the culture medium inlet,
wherein the internal space of the culture vessel is isolated from an atmosphere.

### (Supplementary Note 16)

The culturing system may further comprise a gas supplier configured to supply a gas into the culture vessel, and
a drainer configured to drain the culture medium from the culture vessel.

The wall part may further comprise a gas inlet to which the gas supplier is connected, and a liquid outlet to which the drainer is connected.

### (Supplementary Note 17)

The culturing system may further comprise an observation instrument observing the object cultured in the culture vessel.

### (Supplementary Note 18)

The culturing system may further comprise
a suspension discharger configured to discharge a suspension containing a target cell,
a trap configured to receive the suspension discharged from the suspension discharger and trap the target cell contained in the suspension,
an inducing factor supplier configured to supply an inducing factor to the trap,
an establisher configured to receive the target cell and the inducing factor from the trap and establish an iPS cell, and
a flow channel connecting the establisher and the culture vessel together.

### (Supplementary Note 19)

A method for producing a culture, comprising:
culturing an object while constantly or intermittently supplying a culture medium into a culture vessel,
the culture vessel comprising a rigid bottom part and a wall part, the wall part having a culture medium inlet and comprising a deformable portion, the culture vessel being configured to allow the deformable portion to deform so as to change a capacity of the culture vessel when a pressure of an internal space of the culture vessel varies,
wherein the culturing the object is performed in the culture vessel with the internal space isolated from an atmosphere.

### (Supplementary Note 20)

The object may be an iPS cell.

What is claimed is:

1. A culture vessel comprising:
a rigid bottom part; and
a wall part having a culture medium inlet and comprising a deformable portion,
wherein the culture vessel is configured to allow the deformable portion to deform so as to change a capacity of the culture vessel when a pressure of an internal space of the culture vessel varies.

2. The culture vessel according to claim 1, wherein the bottom part has a light transmitting property.

3. The culture vessel according to claim 1, wherein the deformable portion is an elastic member.

4. The culture vessel according to claim 3, wherein a ratio E1/E2 of an elastic modulus E1 of the deformable portion to an elastic modulus E2 of the bottom part is 0.1 or lower.

5. The culture vessel according to claim 1, wherein the deformable portion is recessed toward the internal space.

6. The culture vessel according to claim 1, wherein the wall part comprises a top part facing the bottom part, and a side part between the bottom part and the top part,
the top part comprising the deformable portion.

7. The culture vessel according to claim 6, wherein the top part further comprises a rigid portion.

8. A culturing system comprising:
a culture vessel comprising a rigid bottom part and a wall part, the wall part having a culture medium inlet and comprising a deformable portion, the culture vessel being configured to allow the deformable portion to deform so as to change a capacity of the culture vessel when a pressure of an internal space of the culture vessel varies; and
a culture medium supplier connected to the culture medium inlet and configured to supply a culture medium into the culture vessel via the culture medium inlet,
wherein the internal space of the culture vessel is isolated from an atmosphere.

9. The culturing system according to claim 8, wherein the bottom part has a light transmitting property.

10. The culturing system according to claim 8, wherein the deformable portion is an elastic member.

11. The culturing system according to claim 10, wherein a ratio E1/E2 of an elastic modulus E1 of the deformable portion to an elastic modulus E2 of the bottom part is 0.1 or lower.

12. The culturing system according to claim 8, wherein the deformable portion is recessed toward the internal space.

13. The culturing system according to claim 8, wherein the wall part comprises a top part facing the bottom part, and a side part between the bottom part and the top part,
the top part comprising the deformable portion.

14. The culturing system according to claim 13, wherein the top part further comprises a rigid portion.

15. The culturing system according to claim 8, further comprising:
a gas supplier configured to supply a gas into the culture vessel; and
a drainer configured to drain the culture medium from the culture vessel,
wherein the wall part has a gas inlet to which the gas supplier is connected, and a liquid outlet to which the drainer is connected.

16. The culturing system according to claim 8, further comprising an observation instrument observing an object cultured in the culture vessel.

17. The culturing system according to claim 8, further comprising:
a suspension discharger configured to discharge a suspension containing a target cell;
a trap configured to receive the suspension discharged from the suspension discharger and trap the target cell contained in the suspension;
an inducing factor supplier configured to supply an inducing factor to the trap;
an establisher configured to receive the target cell and the inducing factor from the trap to establish an induced pluripotent stem cell; and
a flow channel connecting the establisher and the culture vessel together.

18. A method for producing a culture, comprising:
culturing an object in a culture vessel while constantly or intermittently supplying a culture medium into the culture vessel,
the culture vessel comprising a rigid bottom part and a wall part, the wall part having a culture medium inlet and comprising a deformable portion, the culture vessel being configured to allow the deformable portion to deform so as to change a capacity of the culture vessel when a pressure of an internal space of the culture vessel varies,
wherein the culturing the object is performed in the culture vessel with the internal space isolated from an atmosphere.

19. The method according to claim 18, wherein the object is an induced pluripotent stem cell.
